(19) United States
(12) Plant Patent Application Publication
(10) Pub. No.: US 2014/0283247 P1
(43) Pub. Date: Sep. 18, 2014

(54) Variety of Tibouchina named 'Imagine'

(50) ;

(71) Applicant: Terry Keogh, Victoria Point (AU)

(72) Inventor: Terry Keogh, Victoria Point (AU)

(21) Appl. No.: 13/815,692

(22) Filed: Mar. 14, 2013

(51) Int. Cl. A01H 5/00

(52) U.S. Cl. PLT/226

(57) ABSTRACT

‘Imagine’ is a new variety of Tibouchina plant that is suitable for planting in gardens, containers, and in landscaping having mauve/purple flowers that darken through maturity, a domed and dense plant habit, and medium-sized flowers.

### BOTANICAL CLASSIFICATION

Tibouchina mutabilis

### VARIETAL DENOMINATION

‘IMAGINE’

### BACKGROUND OF THE NEW PLANT

The present invention comprises a new and distinct cultivar of Tibouchina plant, botanically known as Tibouchina mutabilis and hereinafter referred to by the cultivar name ‘Imagine’.

The new and ornamental variety was selected by the breeder as a result of an ongoing breeding program that has been conducted by the inventor for more than thirty years at the inventor's nursery in Victoria Point, Queensland, Australia. The purpose of the breeding program is to produce novel varieties of Tibouchina in varying plant sizes, flower colors, and which are well-suited to a range of climate zones. ‘Imagine’ is a hybrid plant derived from the deliberate controlled cross-pollination of the female parent, an individual plant of Tibouchina mutabilis, named ‘Noelene’, and the male parent, an individul plant of Tibouchina mutabilis named ‘Jazzie’. The inventor emasculated flowers of the female parent and applied pollen that was freshly collected from the male parent in 2003. The parent plants were isolated to prevent open pollination. In 2005, the inventor selected ‘Imagine’ as a seedling that had been raised from the cross-pollination described herein. Selection was based on the criteria of habit, flower color, and plant dimensions. The plant was then propagated in 2008 via vegetative cuttings to produce a new generation for final evaluation.

Further, asexual reproductions of ‘Imagine’ at the same location has demonstrated that the combination of characteristics as herein disclosed for the new cultivar are retained and reproduced true to type through more than six successive generations of asexual reproduction.

The color references made herein are to the R.H.S. Colour Chart of The Royal Horticulture Society of London, except where general color terms of ordinary significance are used.

The present invention has not been evaluated under all possible environmental conditions. The phenotype may vary with variation in environment without a change in the genotype of the plant.

The following traits have been repeatedly observed and determined to basic characteristics of ‘Imagine’ which, in combination, distinguish this Tibouchina plant as a new and distinct cultivar:
1. Medium-sized shrub with a domed plant habit at maturity;
2. Dense plant habit;
3. Pale mauve/purple flowers that change to medium mauve/purple towards the margin when the flowers first open—the flowers turn completely medium mauve/purple at maturity;
4. Medium flower size;
5. Large volume of flowers; and
6. Woody, long-lived shrub.

The new variety differs from ‘Noelene’ and ‘Jazzie’ in its smaller, mature plant size. Additionally, the flower color change of ‘Imagine’ as aging occurs is more distinct than with ‘Noelene’.

When compared to ‘Illusion’ (U.S. Plant patent applied for concurrently), ‘Imagine’ has a smaller flower size and plant dimensions. The petals do not overlap as significantly with ‘Imagine’.

When “Imagine’ is compared to ‘Chameleon’, the distinct margin color and predominately white petal color of a newly opened flower are not as prominent as ‘Imagine’. Further, ‘Chameleon’ exhibits more upright mature plant dimensions than ‘Imagine’.

### BRIEF DESCRIPTION OF THE DRAWINGS

The accompanying photographic drawings illustrate the new variety, with the colors being as nearly true as is possible with color illustrations of this type:

FIG. 1 is close-up photograph of the flowers of the new variety; and

FIG. 2 is a photograph of an entire plant of the new variety
Propagation:
Time to initiate roots: About 14-18 days at approximately 21° C.
Time to produce a rooted cutting: About 24-30 days at 21° C.
Plant:
Plant spread: Approximately 27 cm.
Plant height: 40 cm.
Growth rate: Medium to fast.
Lateral branches:
   Length.—Approximately 30 cm.
   Diameter.—Approximately 0.22 cm.
   Quantity.—About 30.
Age of plant when described: Approximately 16 weeks from a rooted cutting.
Growth habit: Upright to bushy.
Height at maturity: 1.5 m.
Width at maturity: 1.5 m.
Stem:
Branching habit: Strong; upright when juvenile.
Main stem diameter: 0.85 cm.
Lateral stem length (average): 15 cm.
Lateral stem width (average): 0.22 cm.
Internode length (range): 1.4 cm to 7.7 cm.
Shape: Square in cross section.
Surface: Rough.
Branch color:
   Juvenile.—N144D.
   Semi-ripe.—146C.
   Mature.—N199D.
Foliage:
Leaf:
   Arrangement.—Opposite.
   Division.—Simple.
   Margin.—Entire.
   Shape.—Elliptic.
   Base.—Cuneate.
   Apex.—Acute to acuminate.
   Surface (adaxial).—Pubescent.
   Surface (abaxial).—Pubescent.
   Texture (adaxial surface).—Weakly ribbed with a matte surface.
   Texture (abaxial surface).—Prominently ribbed.
   Attachment.—Petiolate.
   Length (range excluding petiole).—2.5 cm to 7.5 cm.
   Width (range).—0.85 cm to 2.6 cm.
   Color (adaxial surface).—147A.
   Color (abaxial surface).—146B.
   Color of juvenile foliage not fully expanded.—146C.
Foliar fragrance: None observed to date.
Venation:
   Pattern.—Reticulate; 3 primary parallel veins.
   Color (adaxial surface).—146B.
   Color (abaxial surface).—146C.
   Prominence (adaxial surface).—Depressed.
   Vein prominence (abaxial surface).—Raised.
Petiole:
   Length (range).—0.7 cm to 2.6 cm.
   Diameter (average).—0.13 cm.
   Color.—N144D and 178B.
   Surface.—Pubescent.
   Shape.—Cylindrical, but flattened on the adaxial surface.
Flower:
   Shape.—Rotate.
   Aspect.—Facing upward and outward.
   Inflorescence type.—Solitary.
   Persistent or self-cleaning.—Self-cleaning.
   Blooming period.—Summer through fall; can bloom sporadically during other times.
   Inflorescence fragrance.—None observed to date.
Petals:
   Number.—5.
   Central color (both surfaces).—Pale purple.
   Margin color (both surfaces).—Medium purple.
   Central color as senesces.—Medium purple.
   Margin.—Entire and weakly undulating.
   Apex.—Obtuse to broadly acute.
   Base.—Broadly attenuate.
   Shape.—Obovate to elliptic.
   Surface (abaxial and adaxial).—Glabrous.
   Fused or unfused.—Unfused.
   Petals overlapping.—Absent.
Buds:
   Shape.—Elliptic.
   Apex.—Acute.
Calyx:
   Shape.—Cupule.
Sepals:
   Quantity.—5 in number.
   Margin.—Entire.
   Base.—Truncate.
   Fused or unfused.—Unfused.
Peduncle:
   Shape.—Cylindrical.
Reproductive organs:
Stamens:
   Number.—Usually 10 in number (5 long and 5 short).
   Color at flower first opening.—White.
   Color at flower senescence.—Red/purple.
Anthers:
   Color at flower first opening.—White to very pale purple.
   Color at flower senescence.—Medium purple.
   Shape.—Sickle.
Pollen: None observed.
Pistil:
   Number.—1.
   Shape.—Columnar and curved at the end.
   Color at flower first opening.—White.
   Color at flower senescence.—Pale purple.
Stigma:
   Shape.—Flattened.
Ovary:
   Shape.—Cupule.
   Position.—Inferior.
Seeds: No seeds have been observed to date.

I claim:

1. A new and distinct variety of Tibouchina plant named ‘Imagine’ as herein described and illustrated.
